(19) United States
(12) Patent Application Publication
(10) Pub. No.: US 2004/0138203 A1
(43) Pub. Date: Jul. 15, 2004

(54) Bicyclic lactams and sulfonamides with 5-ht1a -affinity and use thereof for preventing and treating cerebral ischaemia

(76) Inventors: Gerd Steiner, Kirchheim (DE); Kurt Schellhaas, Ludwigshafen (DE); Laszlo Szabo, Dossenheim (DE); Berthold Behl, Limburgerhof (DE); Francisco Javier, Kandel (DE); Lilliane Unger, Ludwigshafen (DE)

Correspondence Address: WOOD, PHILLIPS, KATZ, CLARK & MORTIMER, 500 W. MADISON STREET, SUITE 3800, CHICAGO, IL, 60661, US

(21) Appl. No.: 10/312,813

(22) PCT Filed: Jul. 2, 2001
(86) PCT No.: PCT/EP01/07571

(30) Foreign Application Priority Data
Jul. 3, 2000 (DE) 100 31 391.4

(51) Int. Cl. A61K 31/551; A61K 31/55; A61K 31/496; A61K 31/517

(52) U.S. Cl. 514/217.01; 514/224.2; 514/230.5; 514/218; 514/252.17; 514/253.05; 514/253.06; 514/310; 514/314; 540/593; 540/575; 544/105; 544/284; 544/363; 546/144; 546/176

(57) ABSTRACT

Compounds of the formula I

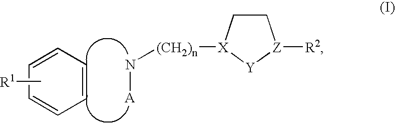

where the substituents possess the meanings given in the description, and their preparation and use as drugs.

The present invention relates to bicyclic compounds of the formula I for the prophylaxis and therapy of cerebral ischemia.

DE 19900544.3 describes bicyclic compounds of the formula 1

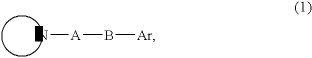

where

A is branched or unbranched (C₁₋₁₀) or straight-chain or branched (C₂₋₁₀)-alkylene which comprises at least one group Z which is selected from O, S, NR⁸, cyclopropyl, CO₂, CHOH or a double or triple bond,

B is 4-piperidine, 4-tetrahydro-1,2,3,6-pyridine, 4-piperazine or the corresponding cyclic compounds which are enlarged by a methylene group, with the linking to A being effected by way of a N atom belonging to B, and

Ar is phenyl, which may be substituted by (C₁₋₆)-alkyl, branched or unbranched, O—(C₁₋₆)-alkyl, branched or unbranched, OH, F, Cl, Br, I, trifluoromethyl, NR²₂, CO₂R², cyano or phenyl, tetralin, indan, higher molecular weight fused aromatic compounds such as naphthalene, which may be substituted by (C₁₋₄)-alkyl or O(C₁₋₄)-alkyl, or anthracene, or 5- or 6-membered aromatic heterocycles which have from 1 to 2 heteroatoms, which are selected, independently of each other, from O and N, and which may be additionally fused to further aromatic residues,

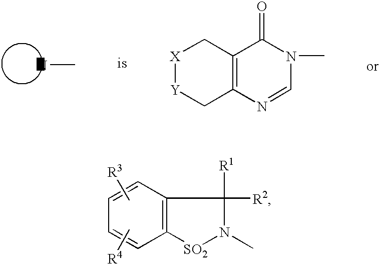

with one of the two radicals X and Y being CH₂ and the other being NR⁹,

R¹,R² are, independently of each other, C₁-C₆-alkyl,

R³ and R⁴ are, independently of each other, hydrogen, (C₁₋₆)-alkyl, branched or unbranched, OH, O—(C₁₋₆)-alkyl, branched or unbranched, F, Cl, Br, I, trifluoromethyl, NR⁵R⁶, CO₂R⁷, nitro, cyano, pyrrole or a phenyl-C₁-C₄ alkyl radical which, for its part, may be substituted on the aromatic moiety by F, Cl, Br, I, C₁-C₄-alkyl, C₁-C₄-alkoxy, trifluoromethyl, hydroxyl, amino, cyano or nitro,

R⁵ and R⁶ are, independently of each other, hydrogen, (C₁₋₆)-alkyl, branched or unbranched, COPh, CO₂tBu or CO—(C₁₋₄)-alkyl, or, together, are a 5- or 6-membered ring which may contain a second N (e.g. piperazine),

R⁷ is hydrogen or (C₁₋₆)-alkyl, branched or unbranched,

R⁸ is hydrogen or C₁-C₄-alkyl,

R⁹ is hydrogen, (C₁₋₆)-alkyl, branched or unbranched, CO—(C₁₋₄)-alkyl, CO₂tBu, CO-aryl or a phenyl-C₁-C₄-alkyl radical which, for its part, may be substituted on the aromatic moiety by F, Cl, Br, I, C₁-C₄-alkyl, C₁-C₄-alkoxy, trifluoromethyl, hydroxyl, amino, cyano or nitro.

Because of their affinity for 5-HT_{1A}, these compounds are suitable for treating cerebral ischemia, in particular stroke.

5-HT_{1A} agonism plays a special role in this context, as can be seen from the studies carried out by SMITHKLINE BEECHAM (EP 345 948), BAYER/TROPON (EP 749 970; De Vry et al., Drugs of the Future 1997, 22 (4), pp. 341-349) and SUNTORY (WO 96/24594, 99/03847).

It has now been found that bicyclic compounds of the formula I

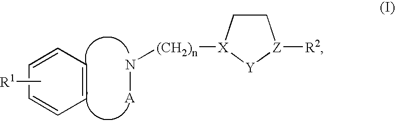

where

the ring containing the increment (N-A) can be 5-, 6- or 7-membered and can additionally also contain an oxygen or sulfur atom or a C—C double bond, with the exception of the 1,4-benzoxazepine skeleton,

A is carbonyl or sulfonyl,

X is nitrogen,

Y is CH₂, CH₂—CH₂, CH₂—CH₂—CH₂ or CH₂—CH,

Z is nitrogen, carbon or CH, with it being possible for the bond between Y and Z also to be a double bond,

n is the number 2, 3 or 4,

R¹ can be hydrogen, halogen, C₁-C₄-alkyl, trifluoromethyl, hydroxyl, C₁-C₄-alkoxy or amino,

R² is phenyl, pyridyl or pyrazinyl which may be monosubstituted or disubstituted by C₁-C₄-alkyl, trifluoromethyl, trifluoromethoxy, hydroxyl, amino, monomethylamino, dimethylamino, cyano or nitro and which can be fused to a benzene nucleus which can be monosubstituted or disubstituted by halogen, C₁-C₄-alkyl, hydroxyl, trifluoromethyl, C₁-C₄-alkoxy, amino, cyano or nitro and may contain 1 nitrogen atom, or to a 5- or 6-membered ring which can contain from 1 to 2 oxygen atoms,

and the physiologically tolerated salts thereof,

are suitable for producing drugs for the prophylaxis and therapy of neurodegeneration, cerebral trauma and cerebral ischemia, in particular stroke, or of the sequelae which are elicited by these diseases.

A use according to the invention also concerns neuroprotection.

Compounds of the formula I which possess a 1,4-benzoxazapine skeleton as depicted below are excluded from the patent claim.

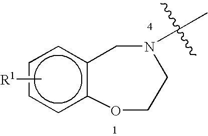

These compounds of the formula I can be prepared by reacting a compound of the formula II

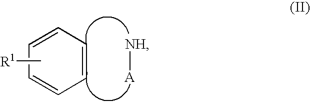

where R¹, A and the ring containing the increment (N-A) have the abovementioned meanings, with a reactive building block of the formula III

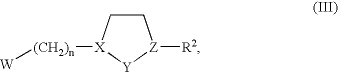

where R², X, Y, Z and n have the abovementioned meanings and W is a leaving group, such as Cl or Br, in the presence of a base, such as sodium hydride or the sodium salt of a lower alcohol or an alkali metal carbonate, and, where appropriate, converting the resulting compound into the acid addition salt of a physiologically tolerated acid.

The reaction expediently takes place in an inert organic solvent, in particular DMF or a lower alcohol, for example methanol or ethanol, or a cyclic, saturated ether, in particular tetrahydrofuran or dioxane, or a hydrocarbon, such as toluene or xylene.

As a rule, the reaction takes place at temperatures of from 20 to 190° C., in particular of from 60 to 90° C., and has generally come to an end within the space of from 1 to 10 hours.

Or else, a compound of the formula II

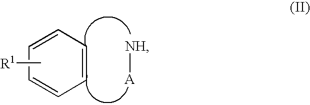

where R¹, A and the ring containing the increment (N-A) have the abovementioned meanings, is reacted with a reactive building block of the formula IV

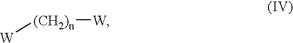

where W is a leaving group, such as Cl or Br, in the presence of a base, with alkali metal hydroxides being preferred, in an inert solvent, preferably halogenohydrocarbons, preferably as a two-phase reaction with water in the added presence of a phase transfer catalyst (aralkyl- or alkylammonium salts), or without solvent in the added presence of an aralkyl- or alkylammonium salt, at temperatures of between 20 and 120° C., to give the cyclization product V.

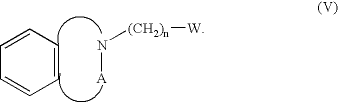

Finally, the halogen derivative of the formula V is reacted with an amine of the general formula VI

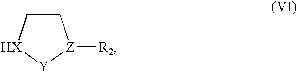

where X, Y, Z and R² have the abovementioned meanings, to give the end product of the formula I according to the invention. This reaction proceeds best in an inert organic solvent, preferably toluene or xylene, in the presence of a base, such as potassium carbonate or potassium hydroxide, at temperatures of between 60 and 150° C.

The compounds of the formula I according to the invention can be purified either by recrystallization from the customary organic solvents, preferably from a lower alcohol, such as ethanol, or by column chromatography.

The free bicyclic compounds of the formula I can be converted, in a customary manner, into the acid addition salts by dissolving them in a solution containing the stoichiometric quantity of the corresponding acid. Examples of pharmaceutically tolerated acids are hydrochloric acid, phosphoric acid, sulfuric acid, methanesulfonic acid, sulfamic acid, maleic acid, fumaric acid, oxalic acid, tartaric acid and citric acid.

The compounds of the formulae II, III, V and VI which are employed as starting materials are known from the literature or can be prepared using analogous protocols from the literature.

The compounds of the present invention possess a surprisingly high affinity for the 5-HT_{1A} receptor, as binding studies carried out using cloned human 5-HT_{1A} receptors showed.

The following test arrangement was used for determining the receptor binding affinity:

5-HT_{1A} binding assay using membranes from 5-HT_{1A} receptor-expressing HEK293 cells

Culturing 5-HT_{1A} receptor-expressing HEK293 cells 5-HT_{1A}-expressing HEK293 cells are cultured, at 37° C. and in a 5% CO₂ atmosphere, in RPMI/Glutamax medium (RPMI 1640, 25 mM Hepes, 2 mM Glutamax, 10% FCS, 2 mM glutamine, penicillin/streptomycin (100 IU/ml each), geneticin G-418 sulfate, 400 mg/l, NaHCO₃, 1.2 g/l) in culture flasks (T-175 triple flasks). After confluence has been reached, the medium is removed and the flasks are filled with 15 ml of sterile PBS (phosphate-buffered saline). The cells are detached by incubating them for 10 minutes (incubator, 37° C.) with a trypsin solution (0.05% trypsin, 0.0004% EDTA, 0.02% EGTA, 2.682 mM KCl, 1.47 mM KH₂PO₄, 6.46 mM NaHPO₄, 136.89 mM NaCl). The detachment of the cells is promoted by knocking on the bottom of the flask. After having been transferred into 50 ml tubes (Greiner), the cells are centrifuged at 250×g at room temperature. The supernatant is discarded and the cells are resuspended in 10 ml of medium. The cells are aliquoted once again into culture flasks and cultured for a further 5 to 6 days until the membranes are prepared.

Preparing the Membranes from 5-HT_{1A} Receptor-Expressing HEK293 Cells

The supernatants are removed from the cells and the culture flasks are filled with PBS. The cells are then incubated for 10 minutes with a trypsin solution (for composition see above). The detachment of the cells is promoted by knocking on the bottom of the flask. The cell suspension is removed and the remaining cells are likewise taken up in PBS by washing the culture flasks twice with PBS. The combined cell suspension is aliquoted into 150 ml Falcon tubes and centrifuged at 250×g and 4° C. for 10 minutes. The supernatants are discarded and the cells in the pellet are resuspended in PBS. 20 μl of the cell suspension are removed and the cell density is determined. The cells are centrifuged once again at 250×g (4° C.) for 10 minutes, after which the supernatant is discarded and the cells in the pellet are homogenized in 50 mM tris-HCl, pH 7.4 (1 ml/10⁸ cells) using an Ultra-Turrax (30 sec).

The homogenate is aliquoted into cryotubes (1 ml/cryotube) and stored in liquid nitrogen until used in the binding assay.

5-HT_{1A} Binding Assay

The frozen membranes are thawed at 37° C., after which they are centrifuged at 48000*g (20 minutes) and then resuspended in binding buffer (50 mM Tris-HCl, pH 7.4, 5 mM CaCl₂). An incubation assay mixture contains membrane material from 50 mg/sample, 0.15 pmol (=0.15 nM) of 3H-8-OH-DPAT, and the substances to be tested, in a total of 1 ml of binding buffer. Nonspecific binding is determined in the presence of 10⁻⁵ M 5-carboxamidotryptamine. Following a 90-minute incubation at 22° C., bound and free ligand are separated from each other by filtering through CF/B filters and subsequently washing with from 5 to 9 ml of ice-cold binding buffer. Before use, the GF/B filters are treated for at least 2 hours with 0.3% polyethylenimine. Following filtration, from 3 to 4 ml of Packard Ultima Gold XR are added to the filters and the radioactivity is determined by liquid scintillation counting in a Packard Tricarb.

Evaluating the Data from the 5-HT_{1A} Binding Assay

The displacement curves are analyzed by nonlinear regression using a modified version of the “Ligand” program of Munson & Rodbard (Anal. Biochem., 107, 220 (1980)). The value of the theoretical nonspecific binding is assessed to be the theoretical binding of radioligand at infinitely high ligand concentration. In this connection, the measured values for the nonspecific binding are treated as data points of the displacement curve which correspond to experimental points at an infinitely high ligand concentration. When testing less than 4 concentrations of a substance or when the specific displacement of the radioligand is <25% (at all the concentrations tested), an IC₅₀ value is estimated using the Hill equation, and the Kᵢ value is calculated in accordance with the equation of Cheng and Prusoff (Biochem. Pharmacol. 22, 3099 (1973)).

The following results (Kᵢ values) are obtained:

| Example | Kᵢ [nM] |
|---|---|
| 1 | 0.6 |
| 2 | 0.6 |
| 3 | 1.6 |
| 4 | 2.6 |
| 5 | 2.9 |
| 6 | 4.1 |
| 7 | 2.4 |
| 8 | 0.1 |
| 10 | 0.5 |
| 13 | 1.0 |
| 19 | 0.7 |
| 22 | 5.4 |

The following examples serve to clarify the invention:

A General Preparation of the Starting Materials of the Formulae III, V and VI

a) 1. 1-[4-(2-Hydroxyethyl)piperazin-1-yl]isoquinoline

47.0 g (350 mM) of 1-(2-hydroxyethyl)piperazine were added to 17.8 g (109 mM) of 1-chloroisoquinoline in 100 ml of ethanol and the mixture was boiled at reflux for 16 h. After it had cooled down, the reaction mixture was partitioned between ethyl acetate and water and the pH was adjusted to 9 with ammonium hydroxide; the aqueous phase was then extracted a further two times with ethyl acetate. After the organic phase had been dried and evaporated, 26.2 g (94%) of product were isolated as a viscous oil.

2. 1-[4-(2-Chloroethyl)piperazin-1-yl]isoquinoline

20.7 g (205 mM) of triethylamine were added to 26.2 g (102 mM) of 1-[4-(2-hydroxyethyl)piperazin-1-yl]isoquinoline in 200 ml of DMF. 23.4 g (205 mM) of methanesulfonyl chloride were then added dropwise, while stirring thoroughly and at room temperature, over a period of 20 min and the reaction mixture was then left to stir for a further 2 h at room temperature. After that, the mixture was partitioned between ethyl acetate and water, with the pH being adjusted to 9 with ammonium hydroxide; the organic phase was then washed thoroughly once again with water. After the organic phase had been dried and evaporated, 26.0 g (93%) of product were isolated as an oil which slowly crystallized throughout. If needed, the product can be further purified by column chromatography (silica gel, eluent ethyl acetate), m.p. from 87 to 89° C.

b) 1-(2-Chloroethyl)-4-(3-trifluoromethylphenyl)piperazine

94.1 g (655 mM) of 1-bromo-2-chloroethane and 43.9 g (434 mM) of triethylamine were added to 100.0 g (434 mM) of 1-(3-trifluoromethyl)piperazine in 1 l of toluene and the reaction mixture was boiled at reflux for 4.5 h. After it had cooled down, the mixture was poured onto ice/water, the organic phase was separated off and the aqueous phase was extracted once again with methyl tert-butyl ether. After the organic phase had been dried and evaporated, 133 g of crude product were isolated, with its crude product then being purified through a silica gel column (eluent heptane/ethyl acetate 1/1). 68.3 g (54%) of pure product were isolated as an oil.

c) 2-(2-Chloroethyl)-2,3-dihydro-1,2-benzisothiazole 1,1-dioxide

A mixture composed of 940 g of 20% strength sodium hydroxide solution and 13.7 g (42.6 mM) of tetrabutylammonium bromide were added dropwise, while stirring thoroughly and over a period of 10 min, to 144.1 g (852 mM) of 2,3-dihydro-1,2-benzisothiazole 1,1-dioxide and 244.2 g (1.7 M) of 1-bromo-2-chloroethane in 25 ml of methylene chloride. The mixture was then left to stir for a further 5 h, after which 400 ml of toluene were added. The organic phase was subsequently washed a further two times with water and the aqueous phase was reextracted twice with 200 ml of toluene on each occasion. After having been dried and evaporated, the combined organic phases yielded the crude product, which was recrytallized from isopropanol. Yield, 142 g (72%), having a m.p. from 88 to 90°C.

d) 2-(2-Chloroethyl)-1-isoindolinone

21.6 g (338 mM) of KOH powder (88%) and 1.0 g (4.39 mM) of triethylbenzylammonium chloride were added to 30.0 g (225 mM) of phthalimidine in 300 ml of 1,2-dichloroethane, and the mixture was boiled at reflux for 3 h. After it had cooled down, 400 ml of water were added and the phases were allowed to separate at pH=7; the aqueous phase was then extracted once again with methylene chloride. After drying, the combined organic phases were evaporated. The crude product was purified by column chromatography (silica gel, eluent methylene chloride/methanol 100/1). 13.3 g (30%) of product, having a m.p. of from 77 to 79° C., were isolated.

e) 1-(4-Trifluoromethyl-2-pyridinyl)-piperazine

93.0 g (1079 mM) of piperazine were added to 28.0 g (154 mM) of 2-chloro-4-trifluoromethylpyridine in 250 ml of ethanol and the reaction mixture was boiled at reflux for 5 h. After it had cooled down, the mixture was evaporated to dryness and the residue was partitioned between ethyl acetate and water; the organic phase was rewashed a further two times with water and evaporated after having been dried. 34 g (95%) of product were isolated as an oil.

f) 1-(3-Trifluoromethylphenyl)-1,4-diazepane

6.68 g (66.7 mM) of 1,4-diazepane (homopiperazine), 0.60 g (2.67 mM) of Pd II acetate, 1.62 g (5.33 mM) of tri-o-tolylphosphine and 6.70 g (62.2 mM) of potassium tert-butoxide were added, under nitrogen and while stirring thoroughly, to 10.0 g (44.4 mM) of 1-bromo-3-trifluoromethylbenzene in 300 ml of xylene and the mixture was boiled at reflux for 16 h. After it had cooled down, the reaction mixture was diluted with methylene chloride and filtered; the filtrate was then evaporated. The residue was partitioned between methyl tert-butyl ether and water, and the organic phase was evaporated after having been dried. The crude product was purified by column chromatography (silica gel, eluent THF/methanol/ammonia 50/50/1). 3.64 g (34%) of product were isolated as an oil.

B) Preparation of the End Products

### EXAMPLE 1

2-[2-(4-(1-Isoquinolinyl)-1-piperazinyl)ethyl]-3,4-dihydro-1(2H)-isoquinolinone×2 HCl×2H₂O

0.2 g (6.8 mM) of sodium hydride (80% strength) was added, under nitrogen and while stirring thoroughly, to 1.0 g (6.8 mM) of 3,4-dihydro-1(2H)-isoquinolinone in 50 ml of dimethylformamide and the mixture was subsequently stirred for 30 min. 1.9 g (6.8 mM) of 1-[4-(2-chloroethyl)piperazin-1-yl]isoquinoline were then added and the reaction mixture was subsequently left to stir at 100° C. for 2 h. After the reaction mixture had cooled down, it was evaporated to dryness and the residue was partitioned between methylene chloride and water; the pH was then adjusted to 9 with dilute sodium hydroxide solution. The aqueous phase was extracted once again with methylene chloride. 2.7 g of crude product were isolated after the organic phase had been dried and evaporated, and this crude product was purified by column chromatography (silica gel, eluent ethyl acetate/methanol 10/1). This resulted in the isolation of 0.9 g (34%) of product, which was dissolved in ether and converted with ethereal hydrochloric acid into the hydrochloride, having a m.p. of from 118 to 120° C.

### EXAMPLE 2

2-[2-(4-(1-Isoquinolinyl)-1-piperazinyl)-ethyl]-1(2H)-isoquinolinone×2 HCl×H₂O

0.35 g (11.7 mM) of sodium hydride (80% strength) was added, under nitrogen and while stirring thoroughly, to 1.45 g (10.0 mM) of 1(2H)-isoquinolinone (isocarbostyril) in 50 ml of dimethylformamide and the mixture was subsequently stirred for 30 min. 2.75 g (10.0 mM) of 1-[4-(2-chloroethyl)piperazin-1-yl]isoquinoline were then added and the reaction mixture was left to stir at 80° C. for 2 h. After it had cooled down, the mixture was evaporated to dryness and the residue was partitioned between ethyl acetate and water and the pH was adjusted to 9 with dilute sodium hydroxide solution. The aqueous phase was extracted once again with ethyl acetate. 5.0 g of crude product was isolated after the organic phase had been dried and evaporated, and this crude product was purified by column chromatography (silica gel, eluent ethyl acetate/ethanol 14/1). This resulted in the isolation of 3.15 g (82%) of product, which was dissolved in ether and converted with ethereal hydrochloric acid into the hydrochloride having a m.p. of from 146 bis 148° C.

### EXAMPLE 3

2-[2-(4-(1-Isoquinolinyl)-1-piperazinyl)-ethyl]-2,3-dihydro-1,2-benzisothiazole 1,1-dioxide×2 HCl

1.5 g (10.8 mM) of finely powdered potassium carbonate and 2.5 g (12.0 mM) of 1-(piperazin-1-yl)isoquinoline were added to 2.5 g (10.8 mM) of 2-(2-chloroethyl)-2,3-dihydro-1,2-benzisothiazole 1,1-dioxide in 30 ml of xylene and the mixture was boiled under reflux for 24 h. After it had cooled down, the reaction mixture was evaporated to dryness and the residue was partitioned between methylene chloride and water at pH=10. The aqueous phase was subsequently extracted once again with methylene chloride. 5.6 g of crude product were isolated after the organic phase had been dried and evaporated, and this crude product was purified by column chromatography (silica gel, eluent methylene chloride/methanol 20/1). This resulted in the isolation of 2.4 g (55%) of product, which was dissolved in ether and converted with ethereal hydrochloric acid into the hydrochloride having a m.p. of from 158 to 168° C.

### EXAMPLE 4

2-[2-(4-(6-Methyl-2-pyridinyl)-1-piperazinyl)-ethyl]-1-isoindolinone

1.55 g (11.25 mM) of finely powdered potassium carbonate and 1.99 g (11.25 mM) of 1-(2-(6-methylpyridyl)piperazine were added to 2.2 g (11.25 mM) of 2-(2-chloroethyl)-1-isoindoline in 30 ml of xylene and the mixture was boiled under reflux for 4 h. After it had cooled down, the reaction mixture was evaporated to dryness and the residue was partitioned between methylene chloride and water. The aqueous phase was subsequently extracted once again with methylene chloride. 4.5 g of crude product were isolated after the organic phase had been dried and evaporated, and this crude product was purified by column chromatography (silica gel, eluent methylene chloride/methanol 30/1). 2.2 g (58%) of product, having a m.p. of from 130 to 132° C., were isolated.

### EXAMPLE 5

1-[2-(4-(3-Trifluoromethylphenyl)-1-piperazinyl)ethyl]-1,3-dihydro-2H-indol-2-one×2 HCl

1.0 g (7.5 mM) of oxindole in 30 ml of toluene was boiled under reflux for 12 h together with 2.2 g (7.5 mM) of 1-(2-chloroethyl)-4-(3-trifluoromethylphenyl)piperazine and 0.55 g (3.75 mM) of finely powdered potassium carbonate. After it had cooled down, the reaction mixture was evaporated to dryness and the residue was partitioned between methylene chloride and water. The aqueous phase was subsequently extracted once again with methylene chloride. 4.3 g of crude product were isolated after the organic phase had been dried and evaporated, and this crude product was purified by column chromatography (silica gel, eluent methylene chloride/methanol 30/1). This resulted in the isolation of 1.9 g (65%) of product, which was dissolved in ether and converted with ethereal hydrochloric acid into the hydrochloride having a m.p. of from 256 to 258° C.

### EXAMPLE 6

1-[2-(4-(1-Isoquinolinyl)-1-piperazinyl)-ethyl]-3,4-dihydro-2(1H)-quinolinone

350 mg (11.7 mM) of 80% sodium hydride were added, under nitrogen and while stirring thoroughly, to 1.5 g (10,2 mM) of 3,4-dihydro-2-quinolinone in 30 ml of dimethylformamide and the mixture was subsequently stirred for 30 min. 2.8 g (10.2 mM) of 1-[4-(2-chloroethyl)-piperazin-1-yl]isoquinoline were then added and the reaction mixture was subsequently left to stir at 80° C. for 3 h. After it had cooled down, the reaction mixture was evaporated to dryness and the residue was partitioned between ethyl acetate and water and the pH was adjusted to 9 with dilute sodium hydroxide solution. The aqueous phase was extracted once again with ethyl acetate. 3.7 g of crude product were isolated after the organic phase had been dried and evaporated, and this crude product was then stirred thoroughly with 20 ml of ethyl acetate, cooled and filtered off with suction. The crystals were subsequently washed with a little ethyl acetate and left to dry in air. This resulted in the isolation of 2.4 g (61%) of product having a m.p. of from 133 to 135° C.

### EXAMPLE 7

1-[2-(4-(1-Isoquinolinyl)-1-piperazinyl)-ethyl]-2(1H)-quinolinone×2 HCl×H₂O

0.25 g (8.3 mM) of sodium hydride (80%) was added, under nitrogen and while stirring thoroughly, to 1.0 g (6.9 mM) of 2-hydroxyquinoline in 25 ml of dimethylformamide and the mixture was subsequently stirred for 1 h. 2.0 g (7.0 mM) of 1-[4-(2-chloroethyl)-piperazin-1-yl]-isoquinoline were then added and the reaction mixture was left to stir at 85° C. for 2 h. After the reaction mixture had cooled down, it was evaporated to dryness and the residue was partitioned between ethyl acetate and water and the pH was adjusted to 8 with dilute sodium hydroxide solution. The aqueous phase was extracted once again with ethyl acetate. 3.4 g of crude product were isolated after the organic phase had been dried and evaporated, and this crude product was purified by column chromatography (silica gel, eluent ethyl acetate/ethanol 14/1). This resulted in the isolation of 2.0 g (75%) of product, which was dissolved in ether/ethyl acetate and converted with ethereal hydrochloric acid into the hydrochloride having a m.p. of from 257 to 259° C.

### EXAMPLE 8

2-[2-(4-(1-Naphthyl)-1-piperazinyl)ethyl]-2,3-dihydro-1,2-benzisothiazole 1,1-dioxide×HCl

1.79 g (12.96 mM) of finely powdered potassium carbonate and 2.73 g (12.96 mM) of 1-(1-naphthyl)piperazine were added to 3.0 g (12.96 mM) of 2-(2-chloroethyl)-2,3-dihydro-1,2-benzisothiazole 1,1-dioxide in 30 ml of xylene and the mixture was boiled under reflux for 5 h. After it had cooled down, the reaction mixture was evaporated to dryness and the residue was partitioned between methylene chloride and water. The aqueous phase was subsequently extracted once again with methylene chloride. 7.2 g of crude product were isolated after the organic phase had been dried and evaporated, and this crude product was purified by column chromatography (silica gel, eluent methylene chloride/methanol 50/1). This resulted in the isolation of 3.5 g (66%) of product, which was dissolved in ether and converted with ethereal hydrochloric acid into the hydrochloride having a m.p. of from 278 to 280° C.

The following were prepared in analogy with examples 1 to 8:

9. 2-[2-(4-(2-Pyridinyl)-1-piperazinyl)ethyl]-2,3-dihydro-1,2-benzisothiazole 1,1-dioxide, m.p. from 98 to 101° C.

10. 2-[2-(4-(6-Methyl-2-pyridinyl)-1-piperazinyl)ethyl]-2,3-dihydro-1,2-benzisothiazole 1,1-dioxide, m.p. from 116 to 119° C.

11. 2-[2-(4-(2-Pyrimidinyl)-1-piperazinyl)ethyl]-2,3-dihydro-1,2-benzisothiazole 1,1-dioxide, m.p. from 132 to 134° C.

12. 2-[2-(4-(4-Trifluoromethyl-2-pyridinyl)-1-piperazinyl)ethyl]-2,3-dihydro-1,2-benzisothiazole 1,1-dioxide, m.p. from 129 to 131° C.

13. 2-[2-(4-(3-Trifluoromethylphenyl)-1-piperazinyl)ethyl]-2,3-dihydro-1,2-benzisothiazole 1,1-dioxide, m.p. from 103 to 105° C.

14. 2-[2-(4-(6-Trifluoromethyl-2-pyridinyl)-1-piperazinyl)ethyl]-2,3-dihydro-1,2-benzisothiazole 1,1-dioxide×HCl, m.p. from 221 to 223° C.

15. 2-[2-(4-(3-Trifluoromethylphenyl)-1,4-diazepan-1-yl)ethyl]-2,3-dihydro-1,2-benzisothiazole 1,1-dioxide×HCl, m.p. from 102 to 104° C.

16. 2-[2-(4-(2-Pyridinyl)-1-piperazinyl)ethyl]-1-isoindolinone, m.p. from 163 to 165° C.

17. 2-[2-(4-(4-Trifluoromethyl-2-pyridinyl)-1-piperazinyl)ethyl]-1-isoindolinone, m.p. from 151 to 153° C.

18. 2-[2-(4-(6-Trifluoromethyl-2-pyridinyl)-1-piperazinyl)ethyl]-1-isoindolinone×HCl, m.p. from 224 to 226° C.

19. 1-[2-(4-(1-Isoquinolinyl)-1-piperazinyl)ethyl]-1,3-dihydro-2H-indol-2-one×2 HCl, m.p. from 213 to 215° C.

20. 1-[2-(4-(6-Trifluoromethyl-2-pyridinyl)-1-piperazinyl)ethyl]-1,3-dihydro-2H-indol-2-one×HCl, m.p. from 263 to 265° C.

21. 1-[2-(4-(1-Isoquinolinyl)-1-piperazinyl)ethyl]-5-chloro-1,3-dihydro-2H-indol-2-one×2 HCl×2H₂O, m.p. from 270 to 272° C.

22. 2-[2-(4-(1-Isoquinolinyl)-1-piperazinyl)ethyl]-1-isoindolinone×2 HCl, m.p. from 256 to 258° C.

23. 1-[2-(4-(1-Isoquinolinyl)-1-piperazinyl)ethyl]-1,3,4,5-tetrahydro-2H-1-benzazepin-2-one×2 HCl×2H₂O, m.p. from 158 to 160° C.

24. 2-[2-(4-(1-Isoquinolinyl)-1-piperazinyl)ethyl]-2,3,4,5-tetrahydro-1H-2-benzazepin-1-one×HCl, m.p. from 149 to 151° C.

25. 3-[2-(4-(1-Isoquinolinyl)-1-piperazinyl)ethyl]-1,3-benzoxazol-2(3H)-one, m.p. from 143 to 145° C.

26. 2-[2-(4-(1-Isoquinolinyl)-1-piperazinyl)ethyl]-1,2-benzisothiazol-3(2H)-one×2 HCl, m.p. from 158 to 160° C.

27. 4-[2-(4-(1-Isoquinolinyl)-1-piperazinyl)ethyl]-2H-1,4-benzoxazin-3(4H)-one×HCl×H₂O, m.p. from 278 to 280° C.

28. 5-[2-(4-(1-Isoquinolinyl)-1-piperazinyl)ethyl]-2,3-dihydro-1,5-benzothiazepin-4(5H)-one×2 HCl×2H₂O, m.p. from 178 to 180° C.

29. 5-[2-(4-(1-Isoquinolinyl)-1-piperazinyl)ethyl]-2,3-dihydro-1,5-benzoxazepin-4(5H)-one

30. 3-[2-(4-(1-Isoquinolinyl)-1-piperazinyl)ethyl]-5-chloro-1,3-benzoxazol-2(3H)-one, m.p. from 110 to 112° C.

31. 4-[2-(4-(1-Isoquinolinyl)-1-piperazinyl)ethyl]-2H-1,4-benzothiazin-3(4H)-one, m.p. from 141 to 143° C.

32. 3-[2-(4-(1-Isoquinolinyl)-1-piperazinyl)ethyl]-3,4-dihydro-1H-2,3-benzothiazine 2,2-dioxide×HCl, m.p. from 198 to 200° C.

We claim:

1. A compound of the formula I
where 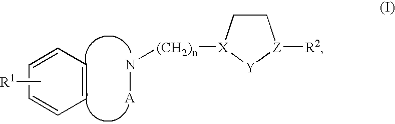
the ring containing the increment (N-A) can be 5-, 6- or 7-membered and can additionally also contain an oxygen or sulfur atom or a C—C double bond, with the exception of the 1,4-benzoxazepine skeleton,
A is carbonyl or sulfonyl,
X is nitrogen,
Y is CH₂, CH₂—CH₂, CH₂—CH₂—CH₂ or CH₂—CH,
Z is nitrogen, carbon or CH, with it being possible for the bond between Y and Z also to be a double bond,
n is the number 2, 3 or 4,
R¹ can be hydrogen, halogen, C₁-C₄-alkyl, trifluoromethyl, hydroxyl, C₁-C₄-alkoxy or amino,
R² is phenyl, pyridyl or pyrazinyl which may be monosubstituted or disubstituted by C₁-C₄-alkyl, trifluoromethyl, trifluoromethoxy, hydroxyl, amino, monomethylamino, dimethylamino, cyano or nitro and which can be fused to a benzene nucleus which can be monosubstituted or disubstituted by halogen, C₁-C₄-alkyl, hydroxyl, trifluoromethyl, C₁-C₄-alkoxy, amino, cyano or nitro and may contain 1 nitrogen atom, or to a 5- or 6-membered ring which can contain from 1 to 2 oxygen atoms,
and the physiologically tolerated salts thereof,

2. The use of compounds as claimed in claim 1 for producing drugs.

3. The use as claimed in claim 2 for the prophylaxis and therapy of neurodegeneration, cerebral trauma and cerebral ischemia, in particular stroke, or of the sequelae which are elicited by these diseases.
